(19) United States
(12) Patent Application Publication
(10) Pub. No.: US 2006/0110326 A1
(43) Pub. Date: May 25, 2006

(54) Toluidine blue o drug substance and use thereof for in vitro staining and chemotherapeutic treatment of dysplastic tissues

(75) Inventor: Karl Okolotowicz, Champagne, IL (US)

Correspondence Address: Drummond & Duckworth, Suite 500, 4590 MacArthur Boulevard, Newport Beach, CA, 92660, US

(73) Assignee: ZILA, INC., Phoenix, AZ (US)

(21) Appl. No.: 10/516,352

(22) PCT Filed: Jun. 4, 2002
(86) PCT No.: PCT/US02/17720

§ 371 Date: Jul. 20, 2005

(51) Int. Cl. A61K 49/00; C07C 245/12

(52) U.S. Cl. 424/9.6; 534/587

(57) ABSTRACT

The invention comprises an improved process for preparing TBO drug products includes the steps: (1) synthesising an indamine; (2) converting said indamine into an S-indaminyl thiosulfate; and (3) adding an oxidizing catalyst agent, complexing agent, and an acid to said S-indaminyl thiosulfate to formulate TBO and C-4-methyl regioisomer, and derivatives thereof. The invention further comprises new compositions of matter that are useful for detecting dysplastic tissue, as well as, treating dysplastic tissue, namely, TBO products predominantly comprised of peaks eight, peak six, or a combination thereof. N,N-dimenthyl-p-phenylenediamine as a starting material results in a TBO product composition comprised of peaks eight, seven, six, and five in the approximate ratios 33:5:5:1, respectively. Whereas N-dimethyl-p-phenylenediamine as a starting material results in a TBO demetylated product composition comprised of peaks six, five, tree and two in the approximate ratio 33:5:5:1, respectively. The invention further comprises an improved PLC method for analyzing the improved TBO drug product, the improvement comprising the addition of an ion-pairing reagent in a first mobile phase and forming a second mobile phase composition comprising 50% alcohol by volume.

The present invention is an improvement upon the Toluidine Blue O compositions, processes and methods disclosed in U.S. Pat. Nos. 6,086,852 and 6,194,573, incorporated herein by reference.

The present invention relates to novel biological stain diagnostic and/or chemotherapeutic compositions that are adapted for human in vivo application.

More particularly, the present invention relates to novel Toluidine Blue O (“TBO”) dye products, which contain TBO and specific TBO derivatives, in specific proportions.

The present invention pertains to new methods of manufacturing TBO compositions, including these novel TBO products.

The present invention further pertains to a new and improved method of HPLC analysis for confirming the compositions of TBO constituents, including these novel TBO products. The improved HPLC process more resolutely separates the HPLC peaks associated with the organic dye content from the peaks associated with degradation products. Additionally, the improved method of HPLC analysis indicates the stability of active organic dye compositions in relationship to degradation products.

The present invention further pertains to in vivo methods of using such novel TBO compositions to identify suspect dysplastic, i.e., abnormal, tissue.

The present invention further pertains to in vivo methods of using such novel TBO compositions as a chemotherapeutic agent against cancerous or precanerous tissue.

In still another and further aspect, the invention pertains to compositions, in vivo diagnostic methods of use thereof, therapeutic treatment methods of use thereof and processes for manufacture thereof, which are specially adapted for use as a chemotherapeutic agent against cancerous and precancerous tissue.

### BACKGROUND OF THE INVENTION

Squamous cell carcinomas usually begin as surface lesions with erythema and slight elevation. These lesions are termed erythroplasia and have been described as early red lesions, which may be either carcinoma in-situ or invasive carcinoma While often asymptomatic, these lesions should be biopsied to determine whether the tissue is malignant or not. Other lesions, called leukoplakia, are pure white. Of these, only 10% of them are found to be carcinoma in-situ or invasive carcinoma.

Common sites for squamous cell carcinoma are the floor of the mouth, the tongue, soft palate, anterior tonsillar pillar, and the retromolar trigone, presumably because the oral tract is often more commonly exposed to carcinogens, such as those found in tobacco. According to recent studies, the depth of the lesion corresponds to a decrease in the percent survival.

| | |
|---|---|
| <2 mm | 95% |
| 2-9 mm | 80% |
| >9 mm | 65% |

Furthermore, patients with early stage oral cancer have a 75% survival at 5 years, but only 35% survival for advanced stages at 5 years. (Emmanuella, J., “Head and Neck Cancer: Squamous Cell Carcinoma”, Medicine Journal, Volume 3, Number 1, Jan. 3, 2002.) Accordingly, early detection and treatment by multiple modalities is important for better prognosis in head and neck cancer.

Epithelial staining is known for facilitating the visual detection of abnormal epithelial cells, granules, denatured epithelial cells, or denatured granules.

In fact, TBO, a dye, is known for early detection, as a guide for optimal biopsy. The dye is absorbed by the mitochondria of malignant cells. As a result, TBO can be used as a screening test and to locate cancer tissue because of its effectiveness in staining malignant and precancerous lesions dark blue without staining normal mucosa. U.S. Pat. No. 4,321,251 to Mashberg and U.S. Pat. No. 5,372,801 to Tucci et al discuss such in vivo diagnostic tests for identifying and delineating suspect dysplastic oral tissue.

Routine examinations entail complete head and neck examination with indirect nasopharyngeal and laryngopharyngeal mirror examination. If an abnormality or suspicious tissue is found, a fine needle aspiration biopsy for cytology or excisional biopsy usually follows. Once a diagnosis of carcinoma is made, endoscopic examination is recommended under general anesthesia with random biopsies of Waldeyer ring, the hypopharynx, nasopharynx, and other common sites of metastasis and any suspicious lesions. General bodywide routine hematologic examinations are encouraged to assess overall medical condition of patient and the possibility of spread to distant organs. (Emmaruella, J., “Head and Neck Cancer: Squamous Cell Carcinoma”, Medicine Journal, Volume 3, Number 1, Jan. 3, 2002.)

In 2001, WIPO Publication Number WO 01/64110 disclosed that TBO is an effective dye for selectively killing cancer cells. Subsequently, WIPO Publication Number WO 01/64255 A1 disclosed that the compounds represented by either peak eight or peak six of the TBO composition disclosed in these WIPO publications, are responsible for the majority of the chemotherapeutic effect on cancer cells. Thus, it is desirable to maximize the production of the compounds represented by the fractions of peak eight and peak six with respect to the other compounds in the drug substance.

### PRIOR ART

The classic synthesis of TBO is thoroughly described in the U.S. Pat. No. 416,055, issued Nov. 30, 1889, to Dandliker et al. This synthesis was described as five sequential steps: (1) oxidation of N,N-dimethyl-p-phenylenediamine, e.g., with potassium dichromate, in the presence of sodium thiosulfate, to form 2-amino-5-dimethylaminophenyl thiosulfonic acid (systematically named “substituted S-phenyl thiosulfate”); (2) condensation of the thiosulfonic acid with o-toluidine, to form the corresponding indamine-thiosulfonic acid (systematically named “substituted S-indaminyl thiosulfate”); (3) ring closure of the indamine-thiosulfonic acid, e.g., in the presence of zinc chloride at boiling temperature for about 30 minutes, to form TBO; (4) the reaction mixture is then cooled and the TBO product of the ring-closure reaction is complexed and salted out, e.g., by treatment with sodium chloride and zinc chloride, to precipitate the TBO complex, e.g., as a TBO/ZnCl₂ complex; and finally, (5) purification may be accomplished by repeated re-solution and re-precipitation, e.g., by re-solution in hot aqueous zinc chloride solution and re-precipitation with sodium chloride/zinc chloride.

These TBO compositions contained a number of impurities, as well as were limited in their organic dye content. For example, the process of manufacture described by Dandliker et al typically yielded compositions that were less than 80% dye content.

HPLC analysis 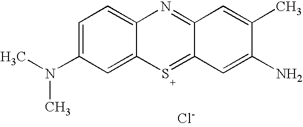
of typical TBO products reveals eight major peaks, symbolizing the compounds representing the components in the composition. True TBO is designated as “peak eight” and is the fraction wherein the molecule is N⁷,N⁷-dimethylated and a methyl group is attached to the C-2, as shown:

### Peak Eight

It is now known that the dye content of typical TBO products included the C-4-methyl regioisomer of TBO (corresponding with peak 7) plus the N-demethylated and N,N-didemethylated derivatives of these substances. Moreover, the N-demethylated derivatives of TBO (corresponding with peaks 5 and 6) and its C-4-methyl regioisomer typically formed greater than 20% of the dye content.

To clarify, other fractions include:

(1) peak seven 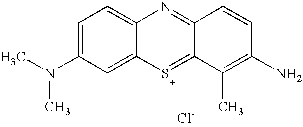
wherein the molecule is N⁷,N⁷-dimethylated and a methyl group is attached to the C-4 as shown:

### Peak Seven

(2) Peaks six and five, which are derivatives formed by N-demethylated of peaks eight and seven, respectively, as shown:

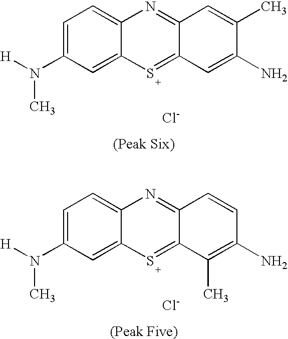

and (3) Peaks three and two, which are further N-demethylated breakdowns of peaks six and five, respectively.

Due to the high percentage of impurities, variability of the dye content and relatively low percentage of true TBO content in the prior art TBO compositions, regulatory approval for human testing of a TBO composition required that manufacturing processes be developed for reproducibly preparing a TBO drug substance with lower impurity content, consistent dye species content, higher total organic dye content, and higher true TBO content.

In developing a regulatory-approvable composition of TBO, Burkett made improvements, as described in U.S. Pat. Nos. 6,086,852 and 6,194,573, to the composition and the method for manufacturing, use, and analysis of TBO drug substances.

Since the compounds represented by peak eight or peak six are now known to possess both the ability to selectively mark as well as selectively kill cancerous and precancerous cells, it would be highly desirable to provide a process to maximize the content of these compounds in a TBO drug substance, without unduly complicating the expense and complexity of the manufacturing procedure.

The present invention solves the above problem by providing TBO products that comprise a maximization of the fractions comprising peak eight and/or peak six, respectively. Accordingly, the present invention is the most desirable composition for use in a method for identifying or treating dysplastic tissues.

I have now discovered such a process, products thereof, and methods of use and analysis of the drug substance product thereof.

The various embodiments of the invention and the practice thereof will be apparent to those skilled in the art, from the following detailed description thereof, taken in conjunction with the drawings.

### BRIEF DESCRIPTION OF THE INVENTION

The closest prior art method for manufacturing TBO comprised the steps of: (1) oxidizing N,N-dimethyl-p-phenylenediamine in a first reaction mixture; (2) introducing a source of sulfur-containing nucleophile into said first reaction mixture, to form a first intermediate, substituted S-phenyl thiosulfate; (3) further oxidizing and condensing said first intermediate with o-toluidine, to form a second intermediate, substituted S-indaminyl thiosulfate; (4) further oxidizing said second intermediate to close the indamine ring thereof, to form a TBO-containing reaction product in a third reaction mixture; (5) introducing a TBO-complexing agent into a reaction mixture before said third mixture is formed; and (6) separating the TBO-containing reaction product from said third reaction mixture. The present invention improves upon the previous method by first oxidizing a starting material in the presence of o-toluidine in a first reaction mixture. The starting material may be either a combination or individually N-dimethyl-p-phenylenediamine and/or N,N-dimethyl-p-phenylenediamine, depending on which peak, peak eight or peak six, or a combination thereof, is desired as the dominant fraction in the final product. For purposes of this document, compositions comprised predominantly of peak eight or six, or a combination thereof, are referred to as “TBO product”.

N,N-dimethyl-p-phenylenediamine as a starting material results in a TBO product composition comprised of peaks eight, seven, six, and five in the approximate ratios 33:5:5:1, respectively.

Whereas N-dimethyl-p-phenylenediamine as a starting material results in a TBO demethylated product composition comprised of peaks six, five, three and two in the approximate ratios 33:5:5:1, respectively.

Thus, oxidation of a reaction mixture containing the starting material, N,N-dimethyl-p-phenylenediamine and/or N-dimethyl-p-phenylenediamine, and o-toluidine occurs before introducing a source of sulfur-containing nucleophile. The above modifications result in a wholly unpredicted composition of TBO product, one that maximizes the production of peaks eight and six, respectively. These compositions have been desirable, yet to date, un-achieved.

With regards to N,N-dimethyl-p-phenylenediamine as the starting material, the present invention concerns a new composition of matter, comprising TBO and its C-4-methyl regioisomer and their N-demethylated derivatives of said isomers. More particularly, HPLC analysis (at 290 nm) of the composition reveals that the ratio of the combined areas of the HPLC peaks representing said isomers to the combined areas of the peaks representing said N-demethylated derivatives being at least about 7:1.

In another embodiment, the composition has a ratio of the area of the HPLC peak representing TBO to the area of the peak representing its C-4-methyl regioisomer of at least about 6:1.

In yet another embodiment, TBO comprises at least 73% by weight of the total organic dye content of said composition.

With regards to N-dimethyl-p-phenylenediamine as the starting material, the present invention concerns a new composition of matter, comprising the N-demethylated derivatives of TBO and its C-4-methyl regioisomer. More particularly, HPLC analysis (at 290 nm) of the composition reveals that the ratio of the combined areas of the HPLC peaks representing said N-demethylated derivatives to the combined areas of the peaks representing said further, N,N-demethylated, derivatives as being at least about 7:1.

In another embodiment, the composition has a ratio of the area of the HPLC peak representing the N-demethylated derivative having the ring methyl group at the C-2 position to the area of the peak representing the N-demethylated derivative having the ring methyl group at the C-4 position of at least about 6:1.

In yet another embodiment, the N-demethylated derivative having the ring methyl group at the C-2 position comprises at least 73% by weight of the total organic dye content of said composition.

In yet another embodiment, the total organic dye content of said composition is comprised of a mixture of predominantly TBO and N-demethylated derivative having the ring methyl group at the C-2 position.

The described composition is important in a method for identification, as well as, chemotherapeutic treatment of dysplastic tissue.

In yet another embodiment of the invention, TBO product is employed in photodynamic therapy to modify the strength of the TBO product's chemotherapeutic treatment effect. In so doing, the incidence of light is controlled in combination with the application of the TBO product. Photodynamic responses in chemotherapeutic treatments has been known since the 1960's as a combination therapy, involving both light and a drug. Upon application of a drug, light is shone and varied by its particular wavelength properties and intensity onto the area to activate or intensify the action of the drug.

The process for manufacturing TBO product includes the steps of: synthesizing an indamine, converting the indamine into an S-indaminyl thiosulfate, further oxidizing the S-indaminyl thiosulfate with an oxidizing agent and lastly complexing the reactant with a complexing agent to formulate a TBO composition. In order to more accurately identify the new composition of TBO product, an improved HPLC method for analysis of a TBO dye product is also disclosed, whereby, the mobile phase has specially adapted a mobile phase composition, and the addition of an ion-pair reagent. The disclosed HPLC method enables separation of HPLC peaks relating to organic dye products from peaks associated with degradation products with an entirely new level of acuity.

The present invention provides a new application of TBO, wherein more pure compositions of TBO product, e.g. peak eight and/or six, is applied to cancer tissues to selectively eliminate or weaken cancer or pre-cancer cells. To our knowledge, moreover, the present invention provides a method for producing the most pure composition of TBO yet manufactured. Consequently, the present invention provides a more pure staining dye for use in clinical procedures for locating cancerous and pre-cancerous tissues.

Additionally, a more sensitive method of HPLC is disclosed for providing an improved resolution in the isolation and identification of TBO and TBO derivative fractions. The improvement builds upon a known BPLC method for analysis of a TBO dye product, which includes: forming a TBO sample solution, forming a mobile phase comprising a water-soluble salt of an organic acid, equilibrating an BPLC column with the mobile phase flow, and injecting the sample solution into the EPLC column. The improvement includes: (1) a forming the mobile phase as a composition including an ion-pair reagent, and (2) forming a second mobile phase composition comprising 50% alcohol by volume.

### BRIEF DESCRIPTION OF THE DRAWINGS

FIG. 1 is a 290 nm BPLC chromatogram, depicting the peaks which are characteristic of the TBO product compositions of the present invention wherein N,N-dimethyl-p-phenylenediamine is the staring material;

FIG. 2 is a 290 nm BPLC chromatogram, depicting the peaks which are characteristic of TBO product compositions previously noted for maximum isolation and production of TBO;

FIG. 3 is a process flow diagram, depicting the preferred embodiment of the invention for manufacturing TBO products, including the novel TBO product compositions of the present invention wherein N,N-dimethyl-p-phenylenediamine is the starting material;

FIG. 4 is the chemical reaction resulting from Step 1 of the process for manufacturing the TBO composition, wherein N,N-dimethyl-p-phenylenediamine is the starting material, depicted by the chemical structures of the reactants and an intermediate;

FIG. 5 is the chemical reaction resulting from Step 2 of the process for manufacturing the TBO composition, wherein N,N-dimethyl-p-phenylenediamine is the starting material, depicted by the chemical structures of the reactants and an intermediate; and

FIG. 6 is the chemical reaction resulting from Step 3 of the process for manufacturing the TBO composition, wherein N,N-dimethyl-p-phenylenediamine is the starting material, depicted by the chemical structures of the reactants and product.

### DETAILED DESCRIPTION OF THE INVENTION

The accompanying drawings illustrate the preferred embodiment of the invention wherein N,N-dimethyl-p-phenylenediamine is the starting material. The drawings should not be construed to limit the invention to exclude N dimethyl-p-phenylenediamine as a starting material, but may be used to provide a model for the very similar reactions wherein N,N-dimethyl-p-phenylenediamine may be reacted to form a TBO product comprised predominantly of peak six.

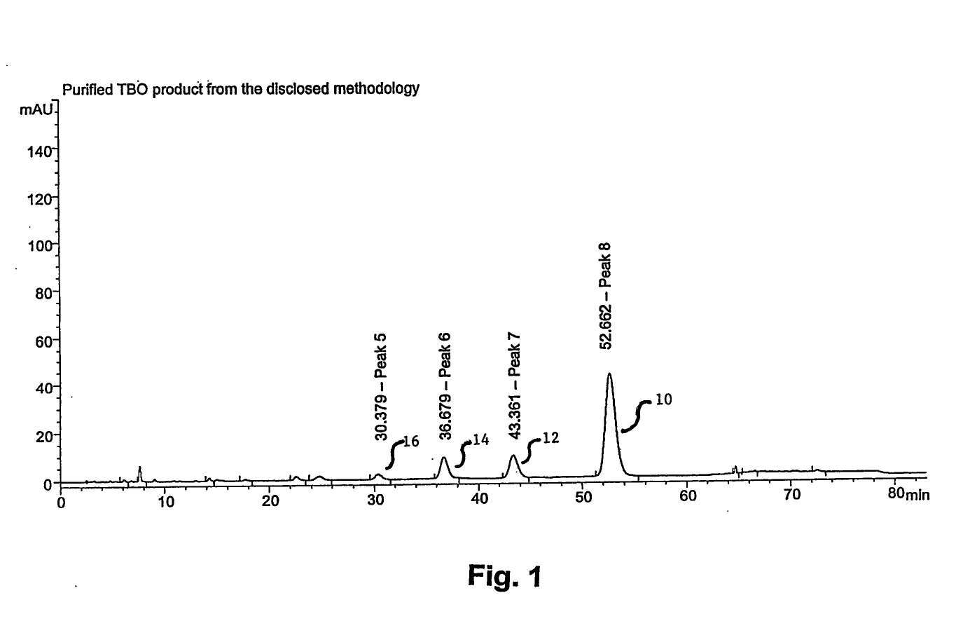

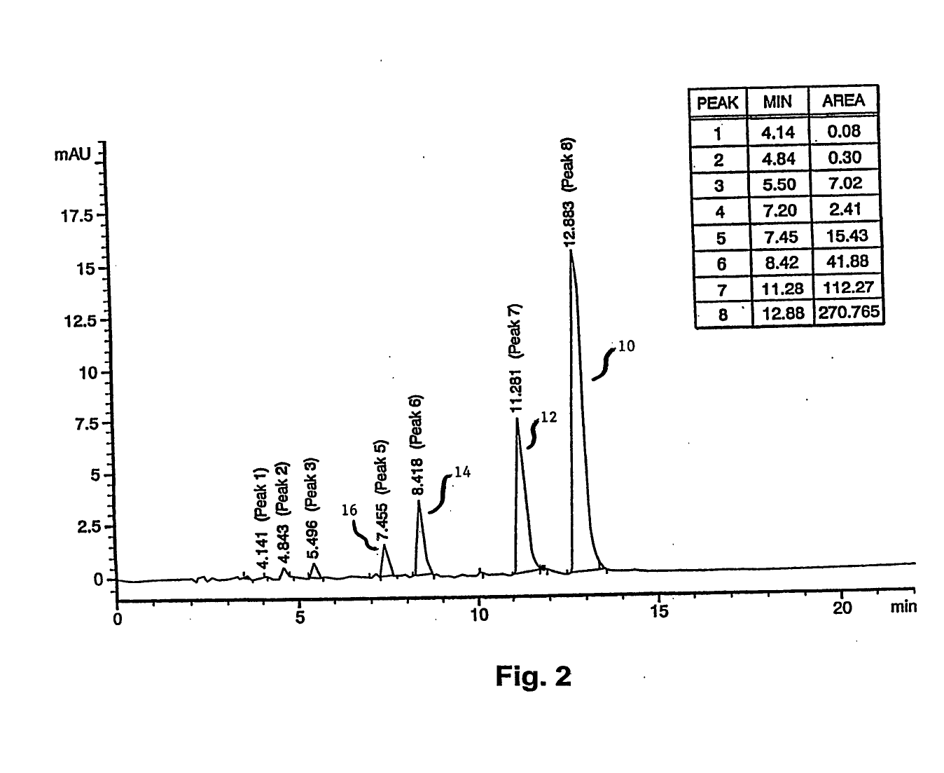

With reference to the accompanying drawings, FIGS. 1 and 2, the present invention for a composition includes the fraction, designated as peak eight 10 (herein referred to as “peak eight”), which achieves a greater percentage by weight of the overall product and a greater percentage in relationship to peaks seven 12, six 14, five 16, three, and two.

More particularly, the present invention concerns the manufacture, and eventual analysis, of a composition having the maximum percent weight of peak eight 10, wherein the fractions of TBO and C-4-methyl regioisomer (peaks eight 10 and seven 12) are seven times the volume by weight of the N-demethylated fractions of TBO and C-4-methyl regioisomer (peaks six 14 and five 16). More particularly, peak eight 10 is produced more than peak seven 12 in a ratio of 6:1.

Peaks eight 10 and six 14 correspond to the TBO, and corresponding N-demethylated derivative, respectively, and wherein the ring methyl group is at the C-2 position. By contrast, peaks seven 12 and five 16 correspond to the C-4-methyl regioisomer of TBO, and corresponding N-demethylated derivative, respectively, and wherein the ring methyl group is at the C-4 position. It is strongly believed that peak eight 10 is more effective than other fractions for staining tissues and for treating cancers with regards to detection and treatment of cancer patients.

A determination of optimal parameters with regards to combining the TBO product mixture with a light during photodynamic therapy is specific to particular cancer types, as well as, patient sensitivity. Using an endoscope or equivalent, the incidence of light can be modified according to wavelength and/or intensity. Accordingly, one skilled in the art is sufficiently informed to determine the optimal light characteristics for combining with the TBO product mixture for optimal drug interaction.

The procedure for manufacturing the present TBO composition is a modification of previously known methods for producing TBO products. More particularly, the modification entails the reversal of the second and third steps described in the process of manufacture described by Burkett in U.S. Pat. No. 6,194,573. The reversal of steps, whereby N,N-dimethyl-p-phenylenediamine and o-toluidine in a stabilized solution are mixed prior to introducing a source of sulfur-containing nucleophile, results in an entirely unexpected composition of TBO. Moreover, the resulting composition, being one that is highly desirable in the detection and treatment of cancer, is an unexpected product to one skilled in the art of chemistry and pharmacology.

Generally, the process entails oxidizing a solution of N,N-dimethyl-p-phenylenediamine and o-toluidine with a stabilizing agent. Following oxidation, an acid is introduced. Then, a complexing agent, an oxidizing agent, and a source of sulfur-containing nucleophile are added. At this point, an intermediate, S-indaminyl thiosulfate 40 is created. The S-indaminyl thiosulfate 40 is then oxidized with an oxidizing agent, followed by the addition of a complexing agent, an oxidizing catalyst agent, and an acid.

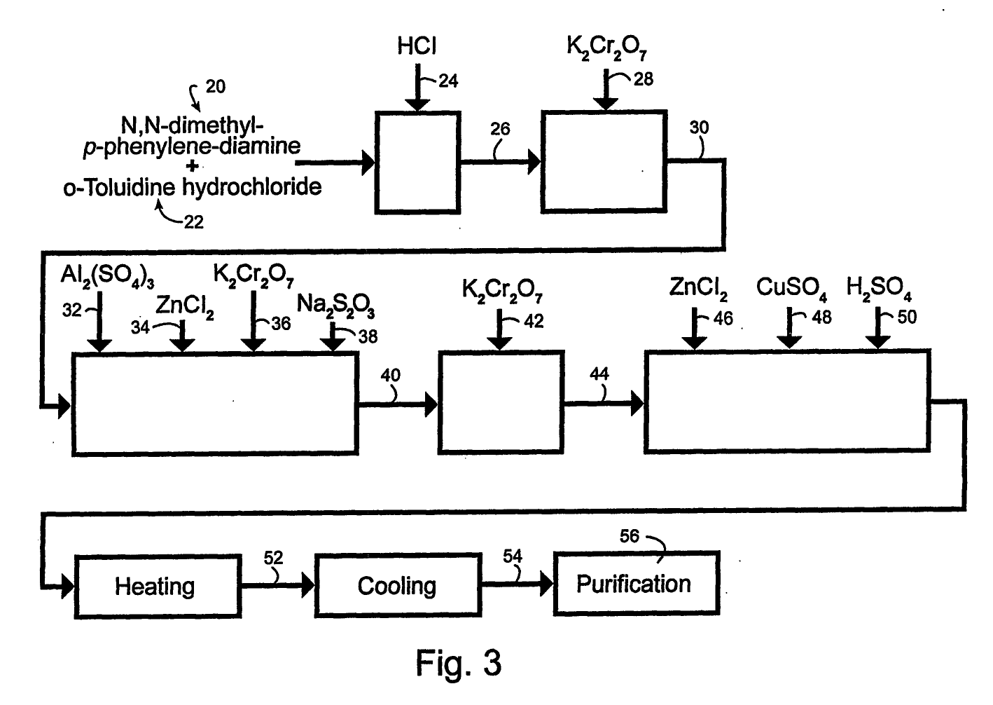

FIG. 3 is provided as a reference for the following steps, labeled Steps 1-4, which describe in detail the preferred embodiment of the present invention concerning the manufacture of a TBO composition.

Step #1—Synthesis of an Indamine

Five grams of NN-dimethyl-p-phenylenediamine dihydrochloride powder is added to 155 g of USP purified water and stirred at 300 rpm in a round-bottom flask. This N,N-dimethyl-p-phenylenediamine reaction mixture 20 should be maintained at ≦10° C. and stirred for 10 minutes.

o-Toluidine hydrochloride is prepared by slowly adding 6.3 grams of hydrochloric acid (6 N) to 2.8 g of o-toluidine. The o-toluidine hydrochloride solution 22 should be stirred until clear, and a minimal amount of USP purified water should be added to redissolve o-toluidine hydrochloride crystals, if crystals appear.

The o-toluidine hydrochloride solution 22 is added to the N,N-dimethyl-p-phenylenediamine dihydrochloride reaction mixture 20, followed by the addition of an additional 2 g of HCl (6 N) 24. The reaction mixture 26 should be maintained at below ˜10° C. and stirred on ice for 45 minutes. By using an addition funnel, 29.25 g of dichromate solution 28, prepared by adding 9.39 grams of potassium dichromate to 108 grams of USP purified water, should be slowly (over 20 minutes) dripped into the o-toluidine hydrochloride and N,N-diethyl-p-phenylenediamine dihydrochloride reaction mixture 26. Once all of the dichromate solution 28 has been added, the indamine dihydrochloride reaction mixture 30 should be maintained at below ˜10° C. and stirred on ice for 60 minutes. It is understood to one skilled in the art that the reaction mixture includes indamine dihydrochloride and derivatives thereof.

It should be understood that presently, hydrochloric acid is believed to be a suitable stabilizing agent for both the starting material (N,N-dimethyl-p-phenylenediamine and/or N-dimethyl-p-phenylenediamine) and o-toluidine. Accordingly, the preceding step describes the components N,N-methyl-p-phenylenediamine dihydrochloride powder and o-toluidine hydrochloride solution in their respective stabilized forms by hydrochloric acid. The present invention, however is not limited to any particular stabilizing agent and may therefore substitute for hydrochloric acid any suitable stabilizing agent, which is understood by one skilled in the art to encompass any one in an array of known stabilizers.

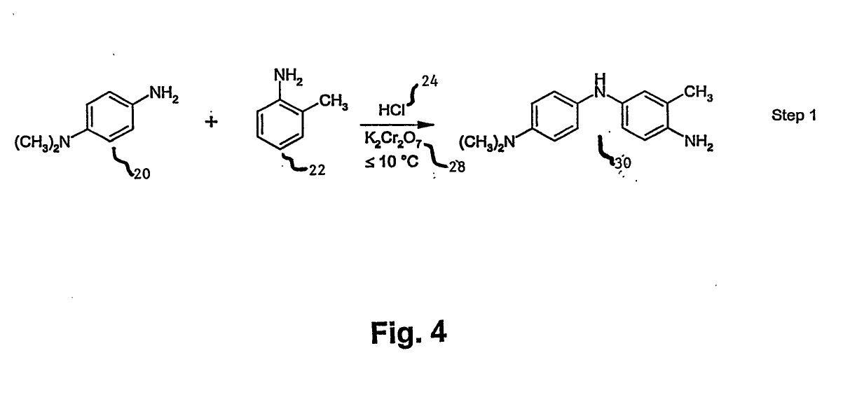

A diagram of the chemical reaction described above in Step 1, is provided in FIG. 4 and demonstrates the reactants and product by their molecular forms.

Step #2—Synthesis of an S-indaminyl thiosulfate

An acid is made by preparing an aluminum sulfate solution 32, which in turn is prepared by adding 8.75 grams of aluminum sulfate hexadecahydrate to 15 grams of USP purified water. This acid is added to the indamine dihydrochloride reaction mixture 30 and stirred for 10 minutes. Acids, other than aluminum sulfate hexadecahydrate, may also be suitable.

A complexing agent is prepared by making a zinc chloride solution 34, which in turn is prepared by adding 12.22 grams of zinc chloride to 15 grams of USP purified water. This complexing agent is added to the indamine dihydrochloride reaction mixture 30 and stirred for 10 minutes. It should be understood that while the preferred embodiment contains zinc chloride, other complexing agents are suitable and embraced by the present invention.

An oxidizing agent is prepared by making 29.25 grams of dichromate solution 36, which in turn is prepared by adding 9.39 grams of potassium dichromate to 108 grams of USP purified water. This oxidizing agent is slowly (over 20 minutes) added, with an addition funnel, to the indamine dihydrochloride reaction mixture 30 and stirred for 20 minutes on ice.

A source of sulfur-containing nucleophile is prepared by making a sodium thiosulfate solution 38, which in turn is prepared by adding 6.53 grams of sodium thiosulfate pentahydrate to 15 grams of USP purified water. This source of sulfur-containing nucleophile is slowly added to the indamine dihydrochloride reaction mixture 30 and stirred on ice for 60 minutes. The precipitate which forms consists of the S-indaminyl thiosulfate 40 and derivatives thereof. As would be understood to one skilled in the art, other sources of sulfur-containing nucleophile may be used and substituted for a sodium thiosulfate solution 38.

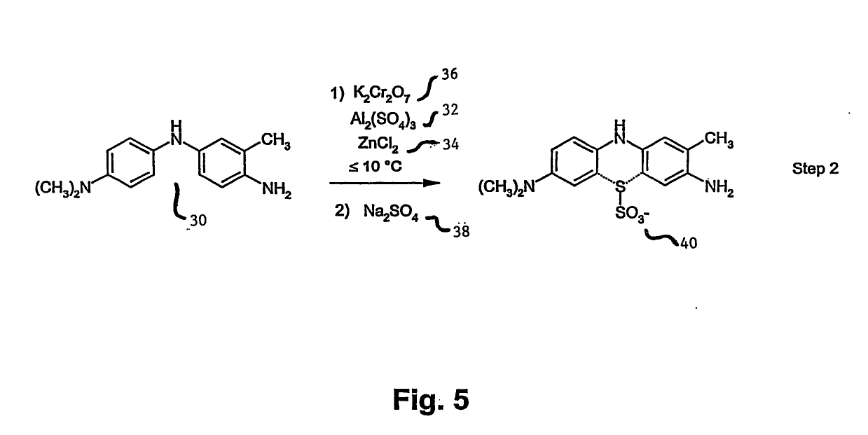

A diagram of the chemical reaction described above in Step 2, is provided in FIG. 5 and demonstrates the reactants and product by their molecular forms. A dotted line can be observed connecting S—SO₃⁻ to two of the carbons. The dotted line signifies that the S—SO₃⁻ may be attached to either carbon.

Step #3—Synthesis of TBO and TBO Zinc Double Salt

The S-indaminyl thiosulfate 40 is maintained below 10° C. With an addition funnel, slowly (over 20 minutes) add an oxidizing agent to the S-indaminyl thiosulfate 40 and stir for 20 minutes. It is presently believed that 29.25 grams of dichromate solution 42 is suitable to be used as an oxidizing agent.

To the oxidized S-indaminyl thiosulfate reaction mixture 44, add a complexing agent, preferably 27.0 grams of zinc chloride solution 46, prepared by adding 12.22 grams of zinc chloride to 15 grams of USP purified water, and stir for 5 minutes. Add an oxidizing catalyst agent, preferably 17.3 grams of copper sulfate solution 48, prepared by adding 2.38 grams copper sulfate to 15 grams of USP purified water, and stir for 5 minutes.

Change the temperature set point to 60° C. Once the reaction reaches 60° C., add an acid, preferably sulfuric acid solution 50 (9 N), to lower pH to 2.9. Stir for 5 minutes after each addition. Change temperature set point to 97° C. Once the reaction mixture reaches 97° C., stir for 35 minutes. Allow reaction mixture 52 to cool to room temperature slowly. Upon reaching room temperature, place the product, which comprises the crude TBO product mixture 54, into a 5° C. cooler. Store for 5-15 hours. Remove and filter an aliquot of the crude TBO product mixture 54 through a 0.45 μm filter. Place filtered solution into a vial for analysis via RP-HPLC TBO analysis method.

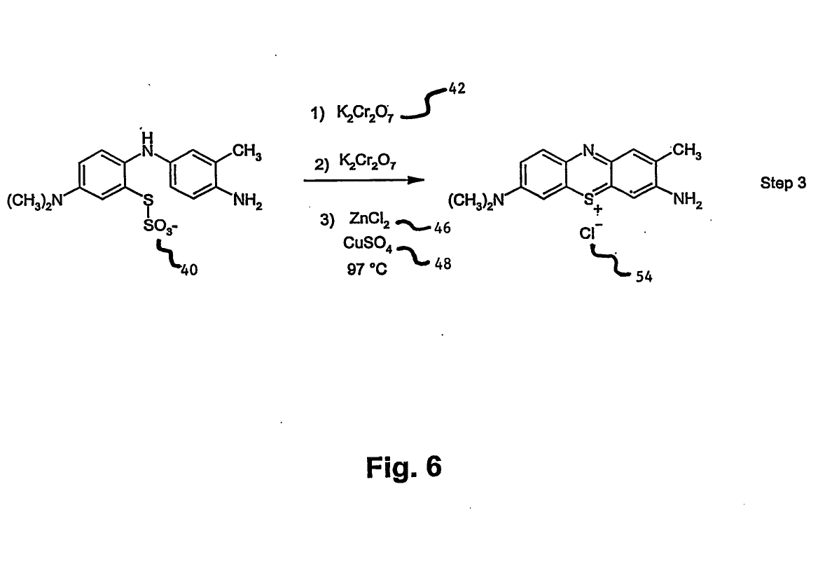

A diagram of the chemical reaction described above in Step 3, is provided in FIG. 6 and demonstrates the reactants and product by their molecular forms.

For clarity, other complexing agents, sulfur-containing nucleophiles, oxidizing agents, oxidizing catalyst agents, and/or acids may be employed, and the preceding steps should be construed as disclosing the complexing agents, reducing agents, oxidizing agents, oxidizing catalyst agents, and/or acids presently believed to be the preferred embodiment of the invention.

Step 4—Purification

It is understood to one skilled in the art that the final reaction mixture contains TBO and C-4-methyl regioisomer, and derivatives thereof. The process for purification 56 of a solution, as described in U.S. Pat. No. 6,194,573, would be known to one skilled in the art, and accordingly, is incorporated herein by reference.

Appropriate laboratory supplies and safety procedures are understood and practiced by those skilled in the art. The laboratory supplies described in U.S. Pat. No. 6,086,852 are suitable to perform the above procedures and, therefore, are incorporated herein by reference.

The above steps for producing TBO differ from previously disclosed methods in that the combining of o-toluidine hydrochloride solution 22 and the starting material, N,N-dimethyl-p-phenylenediamine dihydrochloride solution 20, occurs in the initial step for producing indamine dihydrochloride reaction mixture 30 and prior to the addition of sodium thiosulfate 38. Furthermore, the above steps for producing TBO differs from previously known methods by the addition of hydrochloric acid 24 and potassium chromate 28 before adding any other agents to the reaction mixture of o-toluidine hydrochloride 22 and the starting material, N,N-dimethyl-p-phenylenediamine dihydrochloride 20.

The present invention also encompasses an improved HPLC method for analysis of the improved composition. The improved HPLC method for analysis more resolutely reveals the ratios by weight based upon the area of respective peaks determined by the new HPLC method. The following ratios pertain to a TBO product wherein N,N-dimethyl-p-phenylenediamine is the starting material:
a) Peaks eight 10/Peak seven 12 being approximately 6:1.
b.) Peaks (eight 10+seven 12)/Peaks (six 14+five 16) being approximately 7
c.) Peaks five 16+six 14+seven 12+eight 10 being approximately 95% by weight of the dye content.
d.) Peaks five 16+six 14+seven 12+eight 10 being approximately 75% by weight of the total product including impurities.
e.) Peaks (three+six 14+eight 10)/(two+five 16+seven 12) being approximately 7:1.

Similarly, the following ratios pertain to a TBO product wherein N-dimethyl-p-phenylenediamine is the starting material:
a.) Peak six/Peak five being approximately 6:1.
b.) Peaks (six+five)/Peaks (three+two) being approximately 7:1.
c.) Peaks two+three+five+six being approximately 95% by weight of the dye content.
d.) Peaks two+three+five+six being approximately 75% by weight of the total product including impurities.
e.) Peaks (three+six)/(two+five) being approximately 7:1.

FIGS. 1 and 2 are the respective chromatograms resulting from the improved HPLC method of analysis disclosed, herein, and an analysis of a previously known HPLC method of analysis. An observation of FIGS. 1 and 2 demonstrates the different and comparative flow-gradients taught by each methodology, as well as, the increase in the area of peak eight 10 in relation to the peak seven 12, peak six 14, and peak five 16 in FIG. 1, associated with the present invention. One skilled in the art of chromatography will also observe that the present invention produces superior resolution of TBO product compositions, as well as, being stability indicating. Furthermore, the superior resolution, as demonstrated by the disclosed HPLC analysis, enables the further identification of TBO degradation products. Such identification was, until now, difficult to attain by previous methods.

New Method of HPLC Analysis:

This laboratory method describes the assay procedure for the detection and quantification of the TBO product mixture and derivatives thereof. As would be understood by one skilled in the art of chromatography, various assorted volumetric pipets and flasks are necessary in addition to a column and HPLC analyzer. With regards to the column and BPLC analyzer, a 250×4.6 mm C18 Waters Symmetry Column with 5 μm packing and an HPLC analyzer, HP 1100, 1050 or equivalent are suitable.

The improved HPLC analysis is an improvement upon previously known methods of analysis by adding heptanesulfonic acid, sodium salt as an ion-pair reagent in the Mobile Phase. Here, the ion-pair reagent especially facilitates the separation of TBO compositions. Additionally, heptanesulfonic acid, sodium salt may facilitate the separation of other compounds, especially acidic or cationic compounds. Previously disclosed ion-pair reagents are unable to achieve the resolution between peaks associated with TBO organic dye and peaks associated with degradation products.

Another important improvement to previously known HPLC methods of analysis includes a second mobile phase including an alcohol and a mobile phase solvent in a 50:50 BPLC mobile phase solvent/alcohol Mobile Phase.

Yet, another important improvement includes an adjusted flow rate to 1.0 mL/min., from 1.5 mL/min., to increase the resolution between active peaks and degradation products.

Other specific parameters understood to one skilled in the art of chromatography that optimize the resolution and accuracy of HPLC analysis may vary depending on operating conditions. While these parameters may vary depending on the various circumstances surrounding the conditions of analysis, they are hereby encompassed by the present invention.

While not to be construed to limit the scope of the invention, the preferred embodiment concerning HPLC analysis of TBO products contains the following specific parameters: (1) 290 nm wavelength; (2) 1.0 mL/min flow rate; (3) Injection Volume: 20 μL; (4) temperature: 40° C.; (5) two Mobile Phases:
[A]: 10 mM ammonium acetate @ pH 3.5 with 1.0 g/L 1-heptanesulfonic acid, sodium salt; and
[B]: 50:50 ACN/methanol; and

(6) the following flow gradient:

| Time (min.) | % Mobile Phase B |
|---|---|
| 0.0 | 30.0 |
| 15.0 | 30.0 |
| 20.0 | 29.0 |
| 56.0 | 29.0 |
| 63.0 | 50.0 |
| 75.0 | 50.0 |
| 76.0 | 30.0 |
| 83.0 | 30.0 |

HPLC Preparation:

The following describes a preferred embodiment of an HPLC analysis for analyzing the separate components of a TBO composition. Reagents necessary to perform the improved HPLC method include: purified water, mobile phase solvent (e.g. HPLC grade acetonitrile (ACN)), a buffer salt (e.g. ammonium acetate (CH₃COONH₄)), reagent grade acid (e.g. glacial acetic acid), reagent grade base (e.g. ammonium hydroxide (NH₄OH)), a Secondary Standard (Z97231A.DS) or equivalent, and heptanesulfonic acid, sodium salt.

Mobile Phase A is typically prepared by mixing a suitable buffer salt with H₂O and mixing, preferably approximately 0.77 g of ammonium acetate is dissolved into 1.0 L of H₂O and mixed. The pH is adjusted to 3.5 with an acid or base, for example glacial acetic acid or sodium salt is added and mixed. Finally, the solution is filtered through a 0.45 μm filter.

Mobile Phase B is typically prepared by adding equal volumes of a mobile phase solvent and an alcohol preferably 500 mL of ACN and 500 mL methanol into a 1000 mL graduated cylinder, and mixing. While a volumetric ratio of 50:50 mobile phase solvent to alcohol is prescribed in the preferred embodiment, it should be clear that the present invention encompasses a second Mobile Phase with varying volumetric ratios between mobile phase solvent to alcohol depending on operating conditions and other parameters.

A sample diluent, according to a 90:10 Mobile Phase A: mobile phase solvent solution, is typically prepared by adding 10.0 mL of ACN into 90.0 mL of the pure Mobile Phase A (without heptanesulfonic acid, sodium salt) and mixing. It should be understood that the sample diluent is not limited to comprising ACN, but that various mobile phase solutions known in the art may be suitable.

For analysis, prepare a 0.15 mg/mL sample solution in sample diluent by weighing about 150 mg of TBO product, according to Steps 1-3, into a 100-mL volumetric flask. Dilute to volume with sample diluent and mix. Pipet 10 mL of this initial stock solution into a second 100-mL volumetric flask. Dilute to volume with sample diluent and mix.

A quality control standard sample is prepared by making a 0.15 mg/mL sample solution in sample diluent by weighing about 150 mg of TBO, into a 100.0 mL volumetric flask. Volume is diluted with sample diluent and mixed. 10 mL of this initial stock solution is pipetted into a second 100-mL volumetric flask and diluted to 100.0 mL volume with sample diluent and mixed. Typically, two standard samples are prepared for standard operating procedures and quality control considerations.

In order to assess the HPLC's System suitability, first inject 20 μL of the standard solution into the HPLC to obtain a Capacity Factor (k′) of peak five 16≧peak eight 10. The tailing factor for any active peak (five 16, six 14, seven 12, and eight 10) should be less than or equal to 2.5. Additionally, the resolution between any active peak and its nearest neighbor is not less than 1.0.

Analyze the second standard and evaluate against the working standard for preparation accuracy, the second standard must be between 97.0 and 103.0% of the theoretical concentration.

Make two injections of a standard at the end of the run, ensuring that it must fall within 96.0-104.0% of the initial standard total area.

HPLC Analysis:

First, inject at least five 20-μL portions of a standard solution into the HPLC to obtain total peak area response for active peak areas of ≦2.0% RSD for replicate injections. Next, inject duplicate portions of each sample preparation into the HPLC under the identical chromatographic conditions as used in the standard calibration.

The relative retention time (RRT) of the TBO product mixture's peaks in the sample preparation should correspond to the relative retention time of the TBO product mixture active peaks in the standard preparation. The ratio between the peaks must be within the range of 0.98-1.02.

A guide to calculate % weight/total weight of individual peaks is as follows:
*Peak five 16 [TBO], % w/w=(Cₙ/Cₛₓ)(Rₛₓ/R_{std})(wt. % of peak five 16 in the Standard)

Where:

Cₙ=Concentration in mg/mL, of TBO in the Standard preparation.

Cₛₓ=Concentration in mg/ml of TBO product mixture in the Sample preparation

Rₛₓ=Average peak five 16 area of TBO product mixture in the Sample preparation.

R_{std}=Average peak five 16 area of TBO in the Standard preparation.

*Repeat the calculation for peaks six 14, seven 12, and eight 10 using the corresponding percent by weight of these peaks in the Standard.

A guide to calculate the total weight percent of TBO product mixture by summing the totals of all four major peaks (five 16, six 14, seven 12, eight 10) is as follows:
Total Assay Value=% w/w peak five 16+% w/w peak six 16+% w/w peak seven 12+% w/w peak eight 10

A guide to calculate the Chromatographic Purity (Area % Purity) is as follows:
Area % Purity=[average sum of peaks (five 16, six 14, seven 12, eight 10) in sample]/(average sum of all peaks in sample)×100%

Having described my invention in such terms as to enable those skilled in the art to understan and practice it and, having identified the presently preferred best modes thereof,

I CLAIM:

1. In a method for manufacturing TBO product comprising sequentially the steps of:
(a) oxidizing the starting material N,N-dimethyl-p-phenylenediamine, in a first reaction mixture;
(b) introducing a source of sulfur-containing nucleophile into said first reaction mixture, to form a first intermediate, substituted S-phenyl thiosulfate;
(c) further oxidizing and condensing said first intermediate with o-toluidine, to form a second intermediate, substituted S-indaminyl thiosulfate;
(d) further oxidizing said second intermediate, to form a TBO-containing reaction produ in a third reaction mixture;
(e) introducing a TBO-complexing agent into at least one of said reaction mixtures; and
(f) separating the TBO-containing reaction product from said third reaction mixture;
the improvement comprising sequentially:
(a) oxidizing a starting material, comprised of at least one compound selected from the group consisting of N,N-dimethyl-p-phenylenediamine and N-dimethyl-p-phenylenediamine, in the presence of o-toluidine in a first reaction mixture to form a first intermediate, an indamine, without forming S-phenyl thiosulfate; and then
(b) introducing a source of sulfur-containing nucleophile into said first reaction mixture form a second intermediate, S-indaminyl thiosulfate.

2. A new composition of matter, comprising:
TBO, which has the ring methyl group at the C-2 position, as at least 73% by weight of the total organic dye content of said composition.

3. A process for manufacturing the composition of claim 2 including the steps of:
(a) synthesizing an indamine; and
(b) synthesizing an S-indaminyl thiosulfate;

4. The process of claim 4 wherein said step of synthesizing an indamine further comprises the step of oxidizing a solution of o-toluidine and a solution of N,N-dimethyl-p-phenylenediamine in the presence of an acid and oxidizing agent.

5. A method for identification of dysplastic tissue comprising:
the step of applying to human tissue the TBO product of claim 2.

6. A method for treating dysplastic tissue comprising:
the step of applying to human tissue the TBO product of claim 2.

7. The method for treating dysplastic tissue of claim 6 further comprising:
modifying the incidence of light to control phototoxic effects.

8. The method for treating dysplastic tissue of claim 6 further comprising:
the step of mixing a chemotherapeutic agent with said TBO product of claim 4.

9. A new composition of matter, comprising:
the N-methylated derivative of TBO, in which the N-demethylated derivatives have the ring methyl group at the C-2 position, comprises at least 73% by weight of the total organic dye content of said composition.

10. A process for manufacturing the composition of claim 9 including the steps of:
(a) synthesizing an indamine; and
(b) synthesizing an S-indaminyl thiosulfate;

11. The process of claim 11 wherein said step of synthesizing an indamine further comprises the step of oxidizing a solution of o-toluidine and a solution of N-dimethyl-p-phenylenediamine in the presence of an acid and oxidizing agent.

12. A method for identification of dysplastic tissue comprising:
the step of applying to human tissue the TBO product of claim 9.

13. A method for treating dysplastic tissue comprising:
the step of applying to human tissue the TBO product of claim 9.

14. The method for treating dysplastic tissue of claim 13 further comprising:
modifying the incidence of light to control phototoxic effects.

15. The method for treating dysplastic tissue of claim 13 further comprising:
the step of mixing a chemotherapeutic agent with said TBO product of claim 9.

16. A new composition of matter, comprising:
TBO, which has the ring methyl group at the C-2 position; and
the N-demethylated derivative of TBO having the ring methyl group at the C-2 position;
in which said TBO and said N-demethylated derivative comprises at least 70% by weight of the total organic dye content of said composition.

17. A process for manufacturing the composition of claim 16 including the steps of:
(a) synthesizing an indamine; and
(b) synthesizing an S-indaminyl thiosulfate;

18. The process of claim 17 wherein said step of synthesizing an indamine further comprises the step of oxidizing a solution of o-toluidine and a solution of N,N-dimethyl-p-phenylenediamine and N-dimethyl-p-phenylenediamine in the presence of an acid and oxidizing agent.

19. A method for identification of dysplastic tissue comprising:
the step of applying to human tissue the TBO product of claim 16.

20. A method for treating dysplastic tissue comprising:
the step of applying to human tissue the TBO product of claim 16.

21. The method for treating dysplastic tissue of claim 20 further comprising:
modifying the incidence of light to control phototoxic effects.

22. The method for treating dysplastic tissue of claim 20 further comprising:
the step of mixing a chemotherapeutic agent with said TBO product of claim 16.

23. In an HPLC method for analysis of a TBO dye product, said method including:
(a) forming a TBO sample solution,
(b) forming a mobile phase comprising a water-soluble salt of an organic acid,
(c) equilibrating an BPLC column with the mobile phase flow, and
(d) injecting the sample solution into the HPLC column,
the improvement for identifying sample dye components and for assaying and determining the purity of said sample, said improvement comprising:
forming said mobile phase as a composition including heptanesulfonic acid; and
forming a second mobile phase composition comprising 50% alcohol by volume.
